(19) United States
(12) Patent Application Publication
(10) Pub. No.: US 2022/0324791 A1
(43) Pub. Date: Oct. 13, 2022

(54) P-DIPHENYL COMPOUND DERIVATIVE MIXTURE AND METHOD OF PRODUCING THE SAME

(71) Applicant: HODOGAYA CHEMICAL CO., LTD., Tokyo (JP)

(72) Inventors: Katsumi ABE, Tokyo (JP); Shusaku KOIE, Tokyo (JP); Yuya OKURA, Tokyo (JP); Takuya UEHARA, Tokyo (JP); Hideyuki OTSUKA, Tokyo (JP)

(73) Assignee: HODOGAYA CHEMICAL CO., LTD., Tokyo (JP)

(21) Appl. No.: 17/708,673

(22) Filed: Mar. 30, 2022

(30) Foreign Application Priority Data
Mar. 31, 2021 (JP) 2021-058918

(51) Int. Cl. C07C 211/55; H01L 51/00; H01L 51/50

(52) U.S. Cl. CPC C07C 211/55; H01L 51/0059; H01L 51/5056; H01L 51/5072

(57) ABSTRACT

A mixture containing p-diphenyl compound derivatives represented by the following general formula (1), general formula (2), and general formula (3).

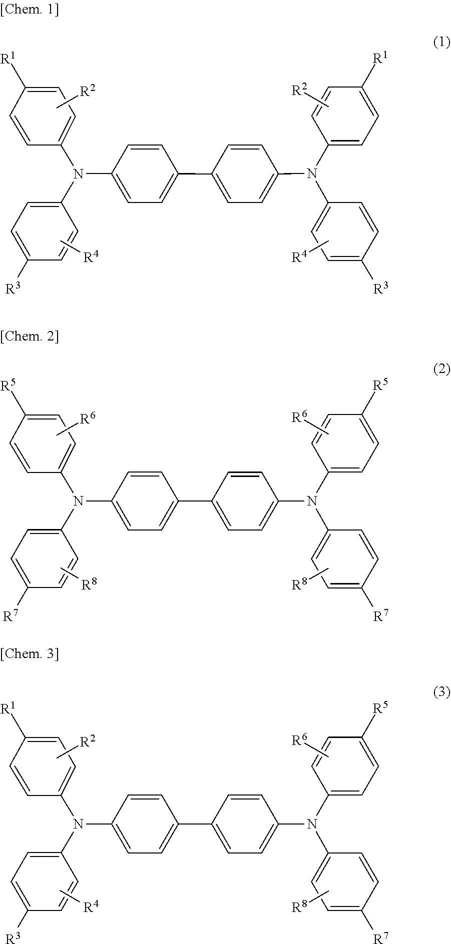

### CROSS REFERENCE TO RELATED APPLICATIONS

This application claims the benefit of Japanese Priority Patent Application JP 2021-058918 filed Mar. 31, 2021, the entire contents of which are incorporated herein by reference.

### BACKGROUND

The present disclosure relates to a p-diphenyl compound derivative mixture and a method of producing the mixture.

In recent years, a device using an organic semiconductor material having charge transportability (charge represents an electron or a hole and the same applies hereinafter) is attracting attention (e.g., Japanese Patent Application Laid-open No. 2012-246484). An organic thin film containing an organic semiconductor material is expected to be applied to an organic electroluminescent device(hereinafter, abbreviated as an organic EL device), a photoelectric conversion device such as a solar battery and an optical sensor, and an organic electronic device such as an organic thin film transistor, and is attractive in terms of light weight, ease of device preparation (increase in the area and flexibility), productivity, low cost, diversity of materials arid functions, and the like, as compared with the case of using an inorganic semiconductor material.

As the organic semiconductor material, particularly, various hole transport materials and electron transport materials that are capable of forming organic thin films have been studied. For example, in order to put the organic EL device to practical use, many improvements have been made to the device structure of the organic EL device, the various roles are further subdivided, and high efficiency and durability are achieved by an electroluminescent device in which an anode, a hole injection layer, a hole transport layer, a light-emitting layer, an electron transport layer, an electron injection layer, and a cathode are sequentially provided on a substrate (e.g., International Publication WO 2008/062636).

In the organic EL device, charges injected from both electrodes are recombined in a light-emitting layer to achieve light emission. Since it is important how efficiently both charges of the hole and the electron can be transferred to the light-emitting layer, the charge transport material plays a large role. For this reason, there is a demand for a charge transport material having performance such as a high charge injection property and large nobility.

Further, the organic thin film containing an organic semiconductor material has been studied in the field of photoelectric conversion applications. For example, a solar battery that includes a photoelectric conversion device using an organic thin film is an artificial device for converting solar energy into electric energy and is important as a technology for effectively utilizing solar energy. Further, a light-receiving device such as an image sensor that is an imaging device is beginning to be used not only for a TV camera and a camera mounted on a smartphone but also for a driving support system, and both the applications and markets are expanding.

However, it is known that the conversion efficiency of these photoelectric conversion devices is generally significantly lower than that in the case of using an inorganic semiconductor. Examples of the cause of lower conversion efficiency in the organic photoelectric conversion device include low quantum yield of carrier light generation, low charge mobility, and high specific resistance, and the development of an organic semiconductor material that contributes to improvement of the conversion efficiency is an issue.

Further, in the development of an organic electronic device using an organic thin film, there is a demand for a material having high, charge mobility as described above, and various reports that the performance of the organic EL device has improved as a result of increasing the purity of the charge transport material for forming an organic thin film have been made (e.g., Japanese Patent Application Laid-open No. 2012-089581). Meanwhile, there is a need to develop a material capable of suppressing the increase in purification cost taken for increasing the purity of a charge transport material, and improving the performance.

A charge transport material that sufficiently satisfies various properties necessary for improving the properties of an organic electronic device as described above has not been obtained at present. Further, at the time of production, although high and stable solubility in a solvent is necessary in the case of performing deposition by a solution process, some materials have not been put to practical use because the solubility in a solvent is poor.

### SUMMARY

It is desired to provide an organic semiconductor compound useful as a charge transport material capable of realizing an organic electronic device than exhibits high solubility for a solution process and favorable electrical properties due to excellent charge mobility.

The present inventors have found a mixture of p-diphenyl compound derivatives containing compounds represented by the following general formula (1), general formula (2), and general formula (3) and a method of producing the mixture, as a result of diligent studies focusing on a p-diphenyl compound. That is, the essence of the present disclosure is as follows.

1. A mixture containing p-diphenyl compound derivatives represented by the following general formula (1), general formula (2), and general formula (3).

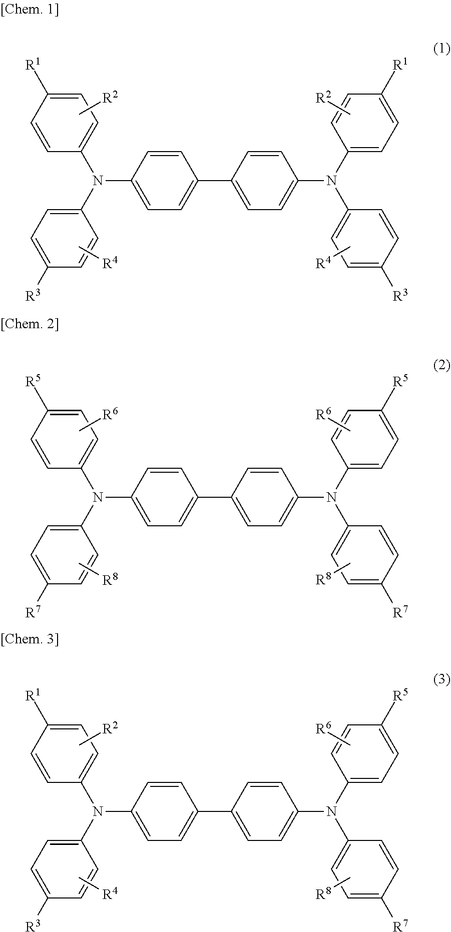

In the formula, R¹ to R⁸ each independently represent

a hydrogen atom, a halogen atom,

a linear or branched alkyl group having 1to 20 carbon atoms, which may have a substituent group,

a linear or branched alkenyl group having 2 to 20 carbon atoms, which may have a substituent group,

a cycloalkyl group having 3 to 10 carbon atoms, which may have a substituent group,

a linear or branched alkoxy group having 1to 20 carbon atoms, which may have a substituent group,

a cycloalkoxy group having 3 to 10 carbon atoms, which may have a substituent group,

an amino group having 1 to 20 carbon atoms, which may have a substituent group, or

an aromatic hydrocarbon group having 6 to 36 carbon atoms, which may have a substituent group, and

R¹ and R², R³ and R⁴, R⁵ and R⁶, and R⁷ and R⁸ may each be bonded to each other to form a ring.

However, the type and/or substitution position of at least one of the substituent groups R⁵ to R⁸ is different from those of the substituent groups R¹ to R⁴. Further, one of R¹ and R³ and one of R⁵ and R⁷ are not hydrogen atoms.]

2. The mixture, in which

in the general formula (1), the general formula (2), and the general formula (3), R¹, R³, R⁵, and R⁷ each represent a hydrogen atom, a linear or branched alkyl group having 1 to 20 carbon atoms, which may have a substituent group, or a linear or branched alkoxy group having 1 to 20 carbon atoms, which may have a substituent group.

3. The mixture, in which

a content ratio of the compound represented by the general formula (1) is 13 to 30.

4. The mixture, in which a content ratio of the compound represented by the general formula (2) is 20 to 32%.

5. The mixture, in which a content ratio of the compound represented by the general formula (3) is 45to 55%.

6. The mixture, in which solubility in 100 g of an organic solvent at room temperature (25±5° C.) is 50 weights or more.

7. A method of producing a mixture containing the compounds represented by the general formula (1), the general formula (2), and the general formula (3) which are obtained by a one-step reaction from compounds represented by the following general formula (4), general formula (5), and general formula (6).

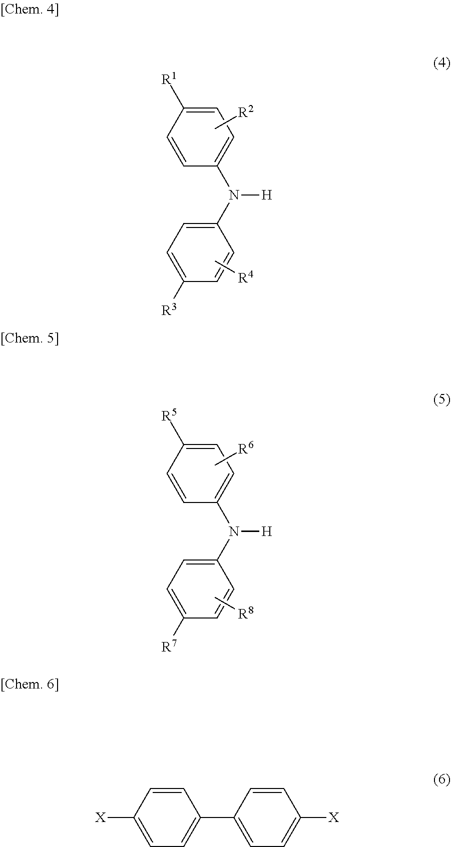

[In the formula, X represents a halogen atom.]

8. The method of producing a mixture, in which

in the general formula (4) and the general formula (5), R₁, R₃, R₅, and R₇ each represent a hydrogen atom, a linear or branched alkyl group having 1 to 20 carbon atoms, which may have a substituent group, or a linear or branched alkoxy group having 1 to 20 carbon atoms, which may have a substituent group.

9. The method of producing a mixture, in which

the one-step reaction is an Ullmann reaction and a reaction temperature is 190 to 235° C.

10. The method of producing a mixture, characterized in that

an additive for the Ullmann reaction is an aromatic oxycarboxylic acid compound.

In accordance with the present disclosure, it is possible to provide a mixture of p-diphenyl compound derivatives that can be used as a charge transport material capable of realizing an organic electronic device that has high solubility in an organic solvent and excellent mobility and exhibits favorable electrical properties.

These and other objects, features and advantages of the present disclosure will become more apparent in light of the following detailed description of best mode embodiments thereof, as illustrated in the accompanying drawings.

### DETAILED DESCRIPTION OF EMBODIMENTS

Hereinafter, an embodiment of the present disclosure will be described in detail.

Although the compounds represented by the general formula (1), the general formula (2), and the general formula (3), which are p-diphenyl compound derivatives according to an embodiment of the present disclosure, will be specifically described, the present disclosure is not limited thereto.

In the general formula (1), the general formula (2), and the general formula (3),

R¹ to R⁸ each independently represent

a hydrogen atom, a halogen atom,

a linear or branched alkyl group having 1 to 20 carbon atoms, which may have a substituent group,

a linear or branched alkenyl group having 2 to 20 carbon atoms, which may have a substituent group,

a cycloalkyl group having 3 to 10 carbon atoms, which may have a substituent group,

a linear or branched alkoxy group having 1 to 20 carbon atoms, which may have a substituent group,

a cycloalkoxy group having 3 to 10 carbon atoms, which may have a substituent group,

an amino group having 1 to 20 carbon atoms, which may have a substituent group, or

an aromatic hydrocarbon group having 6 to 36 carbon atoms, which may have a substituent group.

In the present disclosure, examples of the “halogen atom” include fluorine, chlorine, bromine, and iodine.

In the general formula (1), the general formula (2), and the general formula (3), specific examples of the “linear or branched alkyl group having 1 to 20 carbon atoms” in the “linear or branched alkyl group having 1 to 20 carbon atoms, which may have a substituent group” represented by R¹ to R⁸ include a methyl group, an ethyl group, an n-propyl group, an isopropyl group, an n-butyl group, an isobutyl group, an s-butyl group, a t-butyl group, an n-pentyl group, an isopentyl group, an n-hexyl group, a 2-ethylhexyl group, a heptyl group, an octyl group, an isooctyl group, a nonyl group, and a decyl group, and also include a benzyl group (phenylmethyl group) that is an alkyl group in which one of hydrogen atoms of the alkyl group is substituted with an aromatic hydrocarbon group such as a phenyl group, and a phenethyl group (phenylethyl group).

In the general formula (1), the general, formula (2), and the general formula (3), specific examples of the “linear or branched alkenyl group having 2 to 20 carbon atoms” in the “linear or branched alkenyl group having 2 to 20 carbon atoms, which may have a substituent group” represented by R¹ to R³ include a vinyl group, a 1-propenyl group, an allyl group, a 1-butenyl group, a 2-butenyl group, a 1-pentenyl group, a 1-hexenyl group, an isopropenyl group, an isobutenyl group, and a linear or branched alkenyl group having 2 to 20 carbon atoms to which a plurality of these alkenyl group are bonded.

In the general formula (1), the general formula (2), and the general formula (3), specific examples of the “cycloalkyl group having 3 to 10 carbon atoms” in the “cycloalkyl group having 3 to 10 carbon atoms, which may have a substituent group” represented by R¹ to R⁸ include a cyclopropyl group, a cyclobutyl group, a cyclopentyl group, a cyclohexyl group, a cycloheptyl group, a cyclooctyl group, a cyclodecyl group, and a cyclododecyl group.

In the general formula (1), the general formula (2), and the general formula (3), specific examples of the “linear or branched alkoxy group having 1 to 20 carbon atoms” in the “linear or branched alkoxy group having 1 to 20 carbon atoms, which may have a substituent group” represented by R¹ to R⁸ include a methoxy group, an ethoxy group, a propoxy group, an n-butoxy group, an n-pentyloxy group, an n-hexyloxy group, a heptyloxy group, an octyloxy group, a nonyloxy group, a decyloxy group, an isopropoxy group, an isobutoxy group, an s-butoxy group, a t-butoxy group, an isooctyloxy group, a t-octyloxy group, a phenoxy group, a tolyloxy group, a biphenylyloxy group, a terphenylyloxy group, a naphthyloxy group, an anthryloxy group, a phenanthryloxy group, a fluorenyloxy group, and an indenyloxy group.

In the general formula (1), the general formula (2), and the general formula (3), specific examples of the “cycloalkoxy group having 3 to 10 carbon atoms” in the “cycloalkoxy group having 3 to 10 carbon atoms, which may have a substituent group” represented by R¹ to R⁸ include a cyclopropoxy group, a cyclobutoxy group, a cyclopentyloxy group, and a cyclohexyloxy group.

In the general formula (1), the general formula (2), and the general formula (3), specific examples of the “amino group having 1 to 20 carbon atoms” in the “amino group having 1 to 20 carbon atoms, which may have a substituent group” represented by R¹ to R⁸ include an ethylamino group, an acetyl amino group, a phenylamino group, and the like as monosubstituted amino groups, and a diethylamino group, a diphenylamino group, an acetylphenylamino group, and the like as disubstituted amino groups.

In the general formula (1), the general formula (2), and the general formula (3), specific examples of the “aromatic hydrocarbon group having 6 to 36 carbon atoms” in the “aromatic hydrocarbon group having 6 to 36 carbon atoms, which may have a substituent group” represented by R¹ to R⁹ include a phenyl group, a biphenyl group, a terphenyl group, a naphthyl group, a biphenyl group, an anthraceryl group (anthryl group), a phenanthryl group, a fluorenyl group, an indenyl group, a pyrenyl group, a perylenyl group, a fluoranthenyl group, and a triphenylenyl group. Note that in the present disclosure, the aromatic hydrocarbon group includes a “fused polycyclic aromatic group”.

In the general formula (1), the general formula (2), and the general formula (3), specific examples of the “substituent group” in the “linear or branched alkyl group having 1 to 20 carbon atoms, which may have a substituent group”, “linear or branched alkenyl group having 2 to 20 carbon atoms, which may have a substituent group”, “cycloalkyl group having 3 to 10 carbon atoms. which may have a substituent group”, “linear or branched alkoxy group having 1 to 20 carbon atoms, which may have a substituent group”, “cycloalkoxy group having 3 to 10 carbon atoms, which may have a substituent group”, “amino group having 1 to 20 carbon atoms, which may have a substituent group”, or “aromatic hydrocarbon group having 6 to 36 carbon atoms, which may have a substituent group” represented by R¹ to R⁸ include a halogen atom such as a fluorine atom, a chlorine atom, a bromine atom, and an iodine atom; a cyano group; a hydroxy group; a nitro group; a nitroso group; a carboxyl group; a phosphoric acid group;

a carboxylate ester group such as a methyl ester group and an ethyl ester group;

a linear or branched alkyl group having 1 to 20 carbon atoms, such as a methyl group, an ethyl group, an n-propyl group, an isopropyl group, an n-butyl group, an isobutyl group, an s-butyl group, a t-butyl group, an n-pentyl group, an isopentyl group, an n-hexyl group, a 2-ethylhexyl group, a heptyl group, an octyl group, an isooctyl group, a nonyl group, and a decyl group;

a linear or branched alkenyl group having 2 to 20 carbon atoms, such as a vinyl group, a 1-propenyl group, an allyl group, a 1-butenyl group, a 2-butenyl group, a 1-pentenyl group, a 1-hexenyl group, an isopropenyl group, and an isobutenyl group;

a linear or branched alkoxy group having 1 to 20 carbon atoms, such as a methoxy group, an ethoxy group, a propoxy group, a t-butoxy group, pentyloxy group, and a hexyloxy group;

an aromatic hydrocarbon group having 6 to 30 carbon atoms, such as a phenyl group, a naphthyl group, an anthryl group, a phenanthryl group, and a pyrenyl group;

a heterocyclic group having 5 to 20 ring-forming atoms, such as a pyridyl group, a pyrimidinyl group, a triazinyl group, a thienyl group, a furil group (furanyl group), a pyrrolyl group, an imidazolyl group, a pyrazolyl group, a triazolyl group, a quinolyl group, an isoquinolyl group, a naphthyridinyl group, an acridinyl group a phenanthrolinyl group, a benzofuranyl group, a benzothienyl group, an oxazolyl group, an indolyl group, a carbazolyl group, a benzoxazolyl group, thiazolyl group, a benzothiazolyl group, a quinoxalinyl group, a benzimidazolyl group, a pyrazolyl group, a dibenzofuranyl group, a dibenzothienyl group, and a carbonylyl group;

an amino group having 0 to 20 carbon atoms, such as a monosubstituted amino group such as an unsubstituted amino group (—NH₂), an ethylamino group, an acetyl amino group, and a phenylamino group and a disubstituted amino group such as a diethylamino group, a diphenylamino group, and an acetylphenylamino group; and

a thio group having 0 to 20 carbon atoms, such as an unsubstituted thio group (thiol group:—SH), a methylthio group, an ethylthio group, a propylthio group, a hexa-5-en-3-thio group, a phenylthiol group, and a biphenylthio group. Only one of these “substituent groups” may be contained or a plurality of these “substituent groups” may be contained. In the case where a plurality of these “substituent groups” is contained, the “substituent groups” may be the same as or different from each other. Further, these “substituent group” may further have the substituent group exemplified above.

In the general formula (1), the general formula (2), and the general formula (3), although R¹ to R⁸ each represent the substituent group as described above, R¹ and R², R³ and R⁴, R⁵ and R⁶, and R⁷ and R⁸ may each be bonded to each other via a single bond, a bond via an oxygen atom or a sulfur atom, or a bond via a nitrogen atom to form a ring.

In the general formula (1), the general formula (2), and the general formula (3), the type and/or substitution position of at least one of the substituent groups R⁵ to R⁸ is different from those of the substituent groups R¹ to R⁴. One of R¹ and R³ and one of R⁵ and R⁷ are not hydrogen atoms.

In the general formula (1), the general formula (2), and the general formula (3), from the viewpoint of increasing the charge mobility, R¹, R³, R⁵, and R⁷ each favorably represent a hydrogen atom, a linear or branched alkyl group having 1 to 20 carbon atoms, which may have a substituent group, a linear or branched alkoxy group having 1 to 20 carbon atoms, which may have a substituent group, an amino group having 1 to 20 carbon atoms, which may have a substituent group, or an aromatic hydrocarbon group having 6 to 36 carbon atoms, which may have a substituent group, more favorably represent a hydrogen atom, a linear or branched alkyl group having 1 to 20 carbon atoms, which may have a substituent group, or a linear or branched alkoxy group having 1 to 20 carbon atoms, which may have a substituent group, and still more favorably represent a hydrogen atom or a linear or branched alkyl group having 1 to 20 carbon atoms, which may have a substituent group.

In the general formula (1), the general formula (2), and the general formula (3), from the viewpoint of increasing the charge mobility, R², R⁴, R⁶, and R⁸ each favorably represent a hydrogen atom, a linear or branched alkyl group having 1 to 20 carbon atoms, which may have a substituent group, a linear or branched alkoxy group having 1 to 20 carbon atoms, which may have a substituent group, an amino group having 1 to 20 carbon atoms, which may have a substituent group, or an aromatic hydrocarbon group having 6 to 36 carbon atoms, which may have a substituent group, more favorably represent a hydrogen atom, a linear or branched alkyl group having 1 to 20 carbon atoms, which may have a substituent group, or a linear or branched alkoxy group having 1 to 20 carbon atoms, which may have a substituent group, and still more favorably represent a hydrogen atom or a linear or branched alkyl group having 1 to 20 carbon atoms, which may have a substituent group.

Specific examples of the compounds represented by the general formula (1) and the general formula (2) according to an embodiment of the present disclosure are shown below, but the present disclosure is not limited thereto. Further, the compounds exemplified below are described by omitting some hydrogen atoms and carbon atoms, and the like, show an example of possible isomers, and include all other isomers. Further, these compounds may each be a mixture of two or more kinds of isomers.

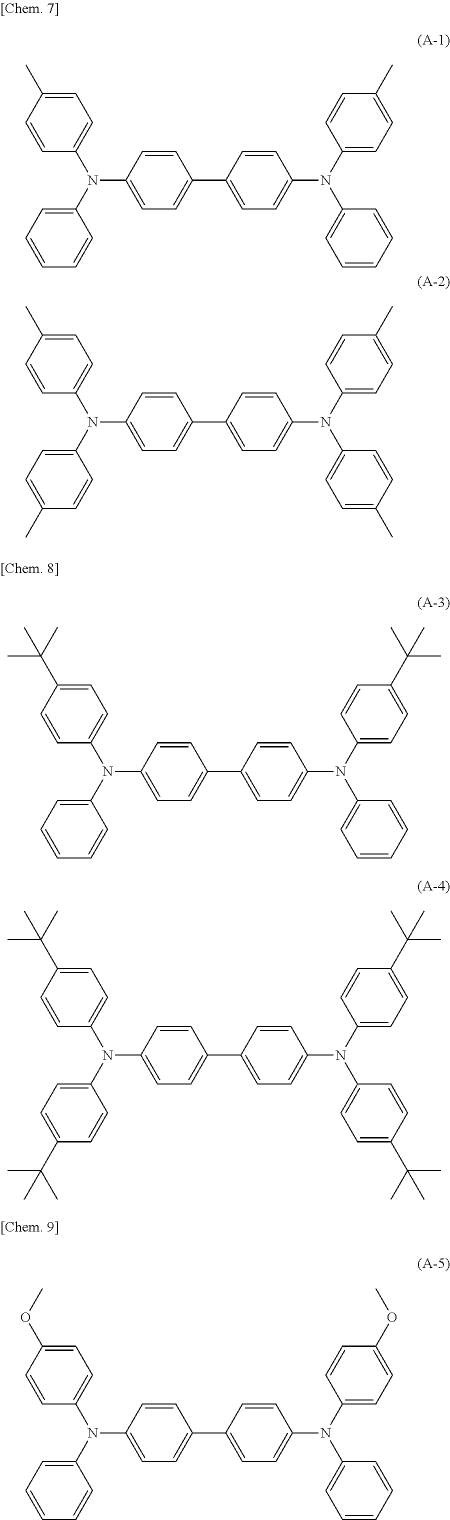

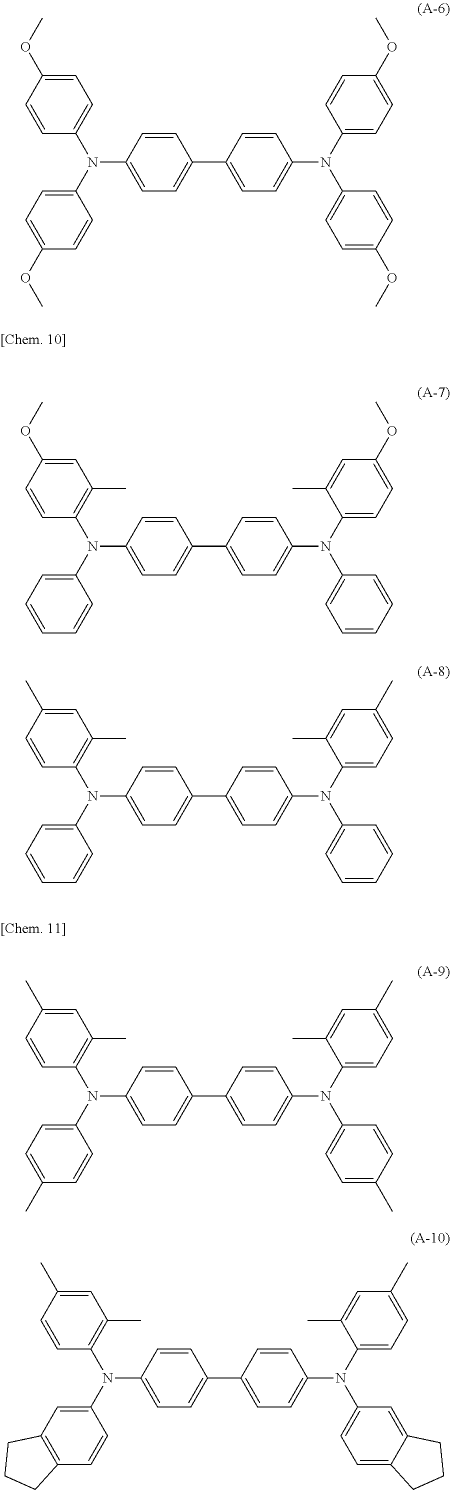 [SMILES: COc1ccc(N(c2ccc(OC)cc2)c2ccc(-c3ccc(N(c4ccc(OC)cc4)c4ccc(OC)cc4)cc3)cc2)cc1 ; COc1ccc(N(c2ccccc2)c2ccc(-c3ccc(N(c4ccccc4)c4ccc(OC)cc4C)cc3)cc2)c(C)c1 ; Cc1ccc(N(c2ccccc2)c2ccc(-c3ccc(N(c4ccccc4)c4ccc(C)cc4C)cc3)cc2)c(C)c1 ; Cc1ccc(N(c2ccc(-c3ccc(N(c4ccc(C)cc4)c4ccc(C)cc4C)cc3)cc2)c2ccc(C)cc2C)cc1 ; Cc1ccc(N(c2ccc(-c3ccc(N(c4ccc5c(c4)CCC5)c4ccc(C)cc4C)cc3)cc2)c2ccc3c(c2)CCC3)c(C)c1]

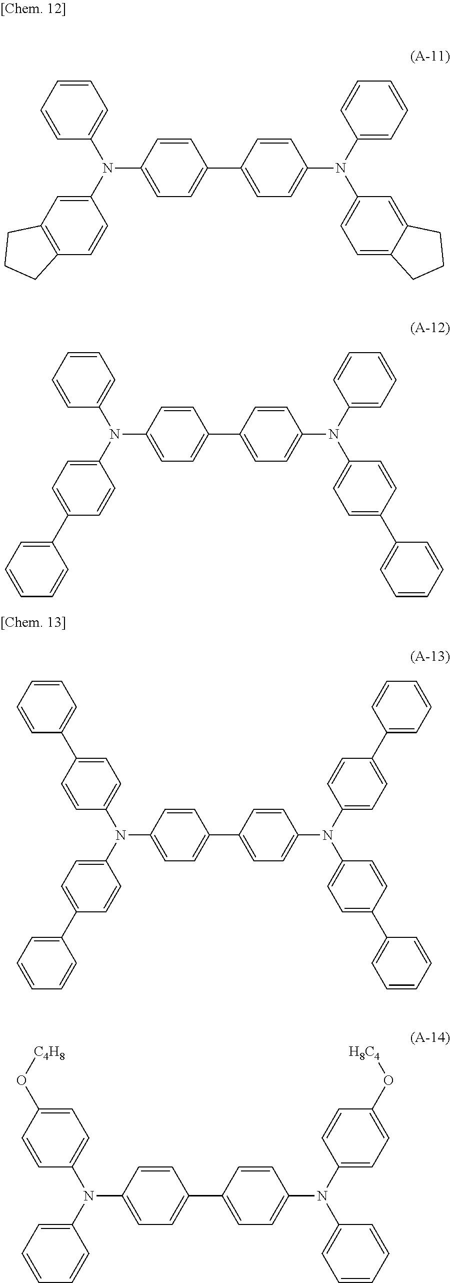

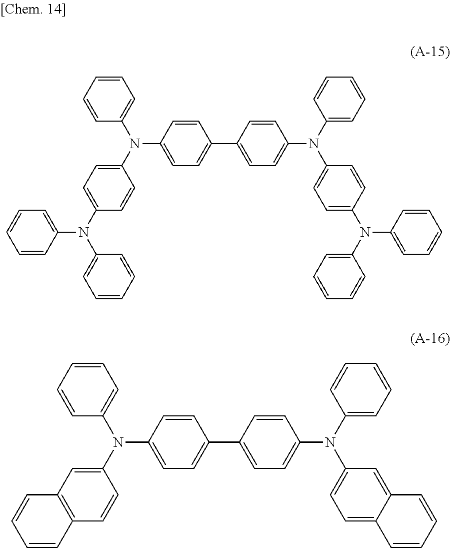 [SMILES: c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

Specific examples of the compound represented by the general formula (3) include the following compounds. Note that the present disclosure is not limited to these compounds.

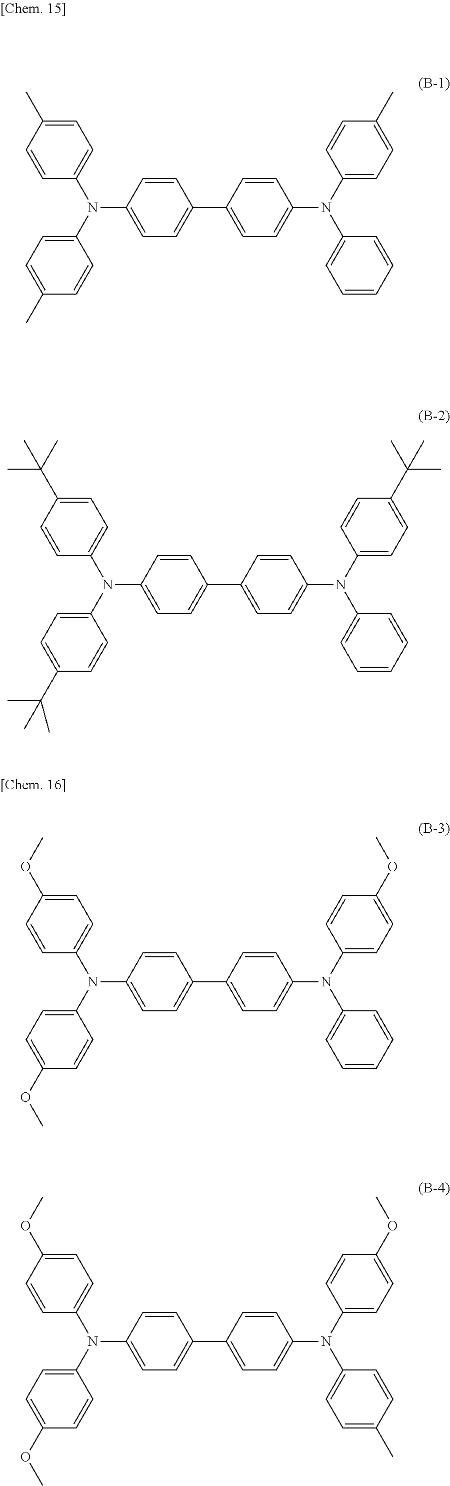

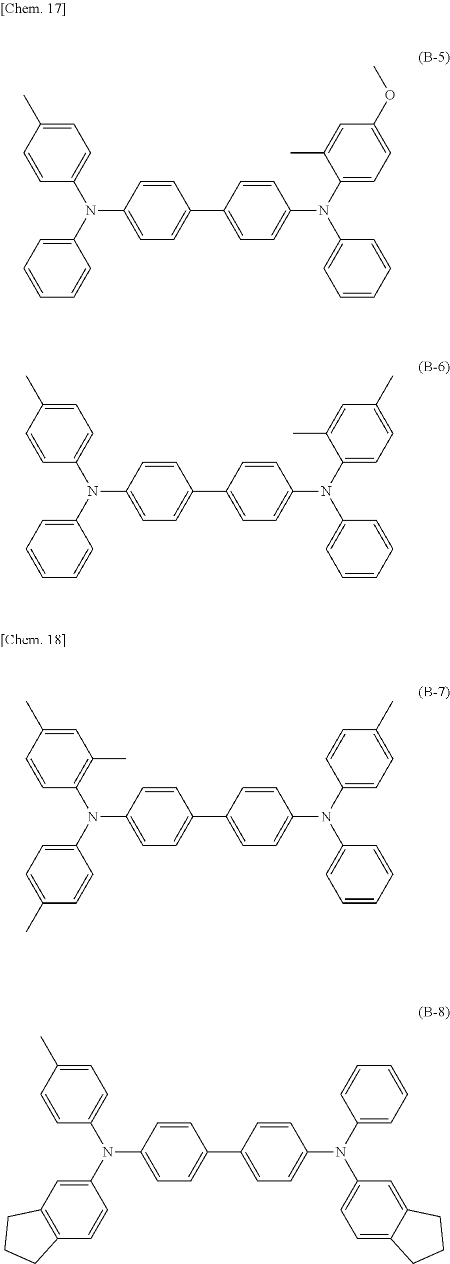

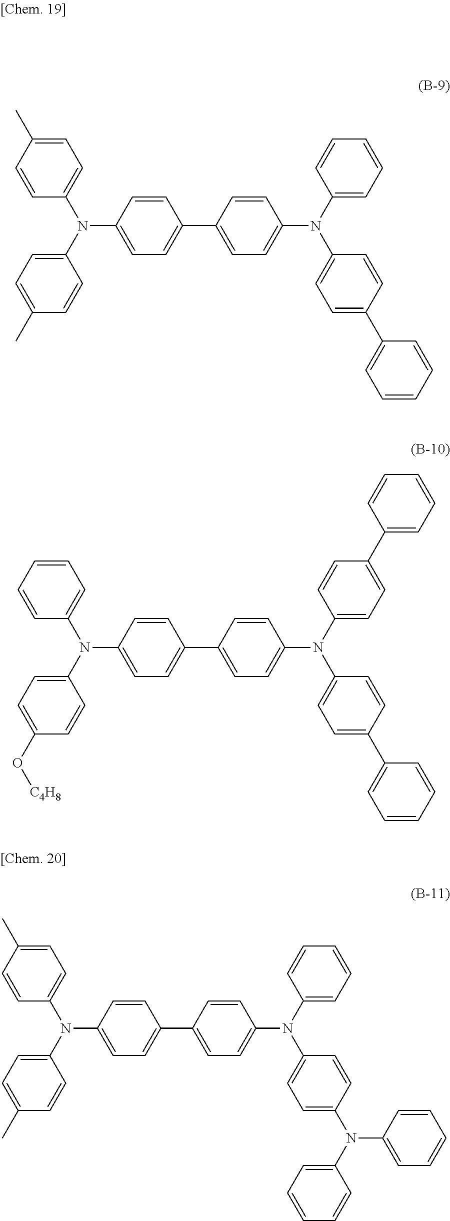

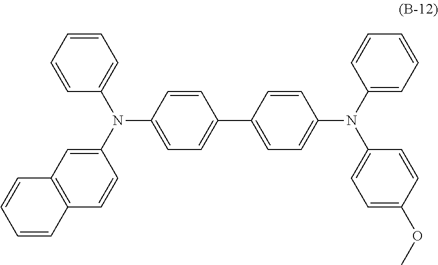

The mixture of the p-diphenyl compound derivatives represented by the general formula (1), the general formula (2), and the general formula (3) according to an embodiment of the present disclosure can be synthesized by a known method such as an Ullmann reaction using a copper catalyst and a base and a Buchwald-Hartwig reaction using a palladium catalyst, but the present disclosure is not limited thereto.

The mixture of the p-diphenyl compound derivatives including the compounds represented by the general formula (1), the general formula (2), and the general formula (3) according to an embodiment of the present disclosure can be synthesized by a one-step reaction of the above reaction from a halogen compound of the p-diphenyl compound represented by the general formula (4), the general formula (6), and the general formula (6).

The R¹ to R⁸ represented by the general formula (4) and the general formula (5) each independently represent

a hydrogen atom, a halogen atom,

a linear or branched alkyl group having 1 to 20 carbon atoms, which may have a substituent group,

a linear or branched alkenyl group having 2 to 20 carbon atoms, which may have a substituent group,

a cycloalkyl group having 3 to 10 carbon atoms, which may have a substituent group,

a linear or branched alkoxy group having 1to 20 carbon atoms, which may have a substituent group,

a cycloalkoxy group having 3 to 10 carbon atoms, which may have a substituent group,

an amino group having 1 to 20 carbon atoms, which may have a substituent group, or

an aromatic hydrocarbon group having 6 to 36 carbon atoms, which may have a substituent group.

In the R¹ to R³ represented by the general formula (4) and the general formula (5), the “linear or branched alkyl group having 1 to 20 carbon atoms, which may have a substituent group”, “linear or branched alkenyl group having 2 to 20 carbon atoms, which may have a substituent group”, “cycloalkyl group having 3 to 10 carbon atoms, which may have a substituent group”, “linear or branched alkoxy group having 1 to 20 carbon atoms, which may have a substituent group”, “cycloalkoxy group having 3 to 10 carbon atoms, which may have a substituent group”, “amino group having 1 to 20 carbon atoms, which may have a substituent group”, or “aromatic hydrocarbon group having 6 to 36 carbon atoms, which may have a substituent group” represents one similar to the “linear or branched alkyl group having 1 to 20 carbon atoms, which may have a substituent group”, “linear or branched alkenyl group having 2 to 20 carbon atoms, which may have a substituent group”, “cycloalkyl group having 3 to 10 carbon atoms, which may have a substituent group”, “linear or branched alkoxy group having 1 to 20 carbon atoms, which may have a substituent group”, “cycloalkoxy group having 3 to 10 carbon atoms, which may have a substituent group”, “amino group having 1 to 20 carbon atoms, which may have a substituent group”, or “aromatic hydrocarbon group having 6 to 36 carbon atoms, which may have a substituent group” represented by R¹ to R⁸ in the general formula (1), the general formula (2), and the general formula (3).

In the R¹ to R⁸ represented by the general formula (4) and the general formula (5), the “substituent group” in the “linear or branched alkyl group having 1 to 20 carbon atoms, which may have a substituent group”, “linear or branched alkenyl group having 2 to 20 carbon atoms, which may have a substituent group”, “cycloalkyl group having 3 to 10 carbon atoms, which may have a substituent group”, “linear or branched alkoxy group having 1 to 20 carbon atoms, which may have a substituent group”, “cycloalkoxy group having 3 to 10 carbon atoms, which may have a substituent group”, “amino group having 1 to 20 carbon atoms, which may have a substituent group”, or “aromatic hydrocarbon group having 6 to 36 carbon atoms, which may have a substituent group” represents one similar to the “substituent group” in the “linear or branched alkyl group having 1 to 20 carbon atoms, which may have a substituent group” or the like represented by R¹ to R⁸ in the general formula (1), the general formula (2), and the general formula (3).

Although the R¹ to R⁸ represented by the general formula (4) and the general formula (5) represents the substituent group as described above, R¹ and R², R³ and R⁴, R⁵ and R⁶, and R⁷ and R⁸ may each be bonded to each other via a single bond, a bond via an oxygen atom or a sulfur atom, or a bond via a nitrogen atom to form a ring.

In the general formula (4) and the general formula (5), the type and/or substitution position of at least one of the substituent groups R⁵ to R³ is different from those of the substituent groups R¹ to R⁴. Further, one of R¹ and R³ and one of R⁵ and R⁷ are not hydrogen atoms.

In the general formula (4) and the general formula (5), R¹, R³, R⁵, and R⁷ each favorably represent a hydrogen atom, a linear or branched alkyl group having 1 to 20 carbon atoms, which may have a substituent group, a linear or branched alkoxy group having 1 to 20 carbon atoms, which may have a substituent group, an amino group having 1 to 20 carbon atoms, which may have a substituent group, or an aromatic hydrocarbon group having 6 to 36 carbon atoms, which may have a substituent group, more favorably indicate a hydrogen atom, a linear or branched alkyl group having 1 to 20 carbon atoms, which may have a substituent group, or a linear or branched alkoxy group having 1 to 20 carbon atoms, which may have a substituent group, and still more favorably represent a hydrogen atom or a linear or branched alkyl group having 1 to 20 carbon atoms, which may have a substituent group.

In the general formula (4) and the general formula (5), R², R⁴, R⁶, and R⁸ each favorably represent a hydrogen atom, a linear or branched alkyl group having 1 to 20 carbon atoms, which may have a substituent group, a linear or branched alkoxy group having 1 to 20 carbon atoms, which may have a substituent group, an amino group having 1 to 20 carbon atoms, which may have a substituent group, or an aromatic hydrocarbon group having 6 to 36 carbon atoms, which may have a substituent group, more favorably represent a hydrogen atom, a linear or branched alkyl group having 1 to 20 carbon atoms, which may have a substituent group, or a linear or branched alkoxy group having 1 to 20 carbon atoms, which may have a substituent group, and still more favorably represent a hydrogen atom or a linear or branched alkyl group having 1 to 20 carbon atoms, which may have a substituent group.

A case of preparing the mixture of the p-diphenyl compound derivatives including the compounds represented by the general formula (1), the general formula (2), and the general formula (3) according to an embodiment of the present disclosure by a one-step reaction of the Ullmann reaction will be described.

In the present disclosure, the copper catalyst is not particularly limited and a known copper catalyst used for the Ullmann reaction can be used. Specific examples of the copper catalyst include a copper powder, copper (I) chloride, copper(II) chloride, copper(I) bromide, copper(II) bromide, copper iodide, copper(I) oxide, copper(II) oxide, copper sulphate, copper nitrate, copper carbonate, copper acetate, and copper hydroxide, and a copper powder, copper(I) chloride, copper(I) bromide, copper iodide, and the like are particularly favorable. In the present disclosure, the amount of the copper catalyst is favorably 0.01 to 1 times mol with respect to 1 mol of the halogen compound of the p-diphenyl compound represented by the general formula (6) used as a raw material and is more favorably within the range of 0.05 to 0.5 times mol.

In the present disclosure, the base is used for dehalogenation and is not particularly limited. For example, alkali metal carbonate salts such as sodium carbonate, lithium carbonate, cesium carbonate, and potassium carbonate, alkali metal phosphate salts such as sodium phosphate, potassium phosphate, cesium phosphate, and lithium phosphate, alkali hydroxide metals such as sodium hydroxide, lithium hydroxide, potassium hydroxide, and cesium hydroxide, alkaline earth metal hydroxides such as barium hydroxide, and metal alkoxides such as sodium methoxide, sodium ethoxide, sodium-tert-butoxide, and potassium-tert-butoxide can be used.

In the present disclosure, the amount of the base used for the reaction is favorably within the range of 1 to 4 times mol with respect to 1 mol of the halogen compound of the p-diphenyl compound represented by the general formula (6) used as a raw material, and is more favorably within the range of 1.5 to 3 times mol.

In the case where the amount of the base is less than the range described above, the yield of the obtained mixture according to an embodiment of the present disclosure decreases. Further, when a large excess of the base than the range described above is added, the post-treatment operation after the reaction is completed becomes complicated, which is not favorable.

In the present disclosure, in the case where the Ullmann reaction is carried out, an additive can be used. As an additive for the Ullmann reaction, for example, diamine compounds such as phenanthroline, bipyridyl, and cyclohexanediamine, and an additive such as 1,1′-binaftil-2,2′-diol and an aromatic oxycarboxylic acid compound can be used in order to obtain a desired product with favorable purity by adding a compound that coordinates with copper of the reaction catalyst to lower the reaction temperature.

In the present disclosure, an aromatic oxycarboxylic acid compound is favorably used. Specific examples of the additive include 2-hydroxybenzenecarboxylic acid, 3-methyl-2-hydroxybenzenecarboxylic acid, 4-methyl-2-hydroxybenzenecarboxylic acid, 5-methyl-2-hydroxybenzenecarboxylic acid, 6-methyl-2-hydroxybenzenecarboxylic acid, 3,5-dimethyl-2-hydroxybenzenecarboxylic acid, 5-ethyl-2-hydroxybenzenecarboxylic acid, 5-propyl-2-hydroxybenzenecarboxylic acid, 5-butyl-2-hydroxybenzenecarboxylic acid, 3-tert-butyl-2-hydroxybenzenecarboxylic acid, 5-tert-butyl-2-hydroxybenzenecarboxylic acid, 3,5-di(tert-butyl)-2-hydroxybenzenecarboxylic acid (3,5-di-tert-butylsalicylic acid), 3-hexyl-2-hydroxybenzenecarboxylic acid, 5-hexyl-2-hydroxybenzenecarboxylic acid, 3-cyclohexyl-2-hydroxybenzenecarboxylic acid, 5-ethenyl-2-hydroxybenzenecarboxylic acid, 5-methoxy-2-hydroxybenzenecarboxylic acid, 5-phenoxy-2-hydroxybenzenecarboxylic acid, 4-nitro-2-hydroxybenzenecarboxylic acid, 4-fluoro-2-hydroxybenzenecarboxylic acid, 5-trifluoromethyl-2-hydroxybenzenecarboxylic acid, 5-cyano-2-hydroxybenzenecarboxylic acid, 2,3-di-hydroxybenzenecarboxylic acid, 2,4-di-hydroxybenzenecarboxylic acid, 2,5-di-hydroxybenzenecarboxylic acid, 4-phenyl-2-hydroxybenzenecarboxylic acid, 5-naphthyl-2-hydroxybenzenecarboxylic acid, 2-hydroxynaphthalene-1-carboxylic acid, and 1-hydroxynaphthalene-2-carboxylic acid. In the present disclosure, 3,5-di(tert-butyl)-2-hydroxybenzenecarboxylic acid (3, 5-di-tert-butylsalicylic acid) is favorably used. By using the additive, side reactions are suppressed and a desired reaction proceeds smoothly, thereby achieving the effects that impurities that are products of side reactions can be reduced and the reaction time can be shortened. Since generation of impurities is suppressed, it is possible to omit the process of purification, which leads to a reduction in production cost.

In the present disclosure, the aromatic oxycarboxylic acid compound is favorably used in an amount of 0.05 to 10 times mol per 1 mol of a copper catalyst, more favorably 0.1 to 5 times mol, and still more favorably 0.2 to 3 times mol.

### Other Additives

In the present disclosure, in order to prevent an oxide from being generated, a sulfite compound or a thiosulfate compound can be added. Examples of the sulfite compound include sodium sulfite, sodium bisulfite, potassium sulfite, potassium bisulfite, magnesium sulfite, cesium sulfite, barium sulfite, and ammonium hydrogen sulfite. Examples of the thiosulfate compound include sodium thiosulfate, sodium dithionite, sodium metabisulfite, ammonium metabisulfite, and potassium metabisulfite.

In the present disclosure, the amount of the sulfite compound or the thiosulfate compound is not particularly limited, but is favorably within the range of 0.01 to 10 times mol, particularly 0.03 to 5 times mol, with respect to 1 mol of the halogen compound of the p-diphenyl compound represented by the general formula (6) used as a raw material.

The Ullmann reaction is generally carried out in a solvent, but can be carried out without a solvent. In general, when the amount of a solvent used is small, the reaction system becomes non-uniform, unreacted substances and the like increase, and the yield decreases in some cases. The reaction according to an embodiment of the present disclosure can be carried out without a solvent or with a small amount of solvent after the reaction is started. The reaction solvent is not particularly United as long as it does not inhibit the reaction. Examples of the reaction solvent include aliphatic hydrocarbon solvents such as octane, nonane, decane, undecane, dodecane, tridecane, and tetradecane, aromatic hydrocarbon solvents such as toluene, xylene, mesitylene, ethylbenzene, diethylbenzene, diisopropylbenzene, hexylbenzene, octylbenzene, dodecylbenzene, methylnaphthalene, dimethylnaphthalene, 1,2,3,4-tetrahydroriaphthalene, and nitrobenzene, ether solvents such as 1,4-dioxane, anisole, and diphenyl ether, amide solvents such as dimethylformamide, dimethylacetamide, N-methylpyrrolidone, and 1,3-dimethyl-2-imidazolidinone, and sulfoxide solvents such as dimethylsulfoxide and tetrahydrothiophene-1,1-dioxide. These may be used alone or two or more of them may be mixed and used.

In the present disclosure, in the case where the Ullmann reaction is carried out, the reaction temperature is favorably 190 to 235° C., more favorably 200 to 230° C., and still more favorably 210 to 225° C., from the viewpoint of suppressing impurities generated by side reactions.

In the present disclosure, the reaction can be carried out under normal pressure or under pressure, and is generally favorably carried out under stirring and under an atmosphere of an inert gas such as nitrogen and argon. Further, in the case where the reaction is carried out under the reaction conditions described above, the reaction time is favorably within the range of 2 to 72 hours from the start of the reaction, and more favorably within the range of 4 to 30 hours.

As the method of purifying the mixture of the p-diphenyl compound derivatives according to an embodiment of the present disclosure, purification with column chromatography, adsorption purification with silica gel, activated carbon, activated clay, or the like, or crystallization such as recrystallization or reprecipitation with a solvent can be used. Alternatively, it is effective to use a compound having increased purity by using these methods. Further, identification of these compounds is performed by performing a single synthesis for each compound and comparing the retention time (RT) by high performance liquid chromatography (wavelength of 254 nm), and identification of the one obtained by the single synthesis is performed by elemental analysis, IR analysis, and NMR analysis. For the mixture, identification of a compound was performed and the mixing ratio was measured by high performance liquid chromatography measurement. From the area ratio (HPLC area ratio) of the component peaks obtained for the respective retention times, the composition ratio (content ratio [%]) of the individual component in the mixture can be known.

It is conceivable that the mixture according to an embodiment of the present disclosure contains an impurity that is a by-product generated by a reaction. Examples of the conceivable impurities include those derived from the general formula (1), those derived from the general formula (2), and those derived from the general formula (3). Examples of those derived include oxidants. Other examples include a dehalogenated compound in which a halogen compound of the p-diphenyl compound represented by the general formula(6) in the reaction process and the diphenylamine compound represented by the general formula (4) or the general formula (5) reacted with each other and a halogen atom of the p-diphenyl compound of the general formula (6) was eliminated.

In order to improve the electrical properties of an organic semiconductor material, the content of impurities that are by-products generated by a reaction is favorably 0 to 5% or less, more favorably 0 to 2% or less, and still more favorably approximately 0 to 1.2% or less.

### Measurement of Content Ratio of By-Product in Mixture

The content ratio of a by-product in the mixture according to an embodiment of the present disclosure can be calculated on the basis of the UV absorption peak area by high performance liquid chromatography (HPLC) and UV light of 254 nm is used to measure the UV absorption peak area. It is conceivable that also the above-mentioned detection components can be similarly detected because they are analog compounds derived from the p-diphenyl compound derivatives according to an embodiment of the present disclosure, which are the main products, and there is no decisive difference in spectroscopic properties.

In the present disclosure, the content ratio [%] (or HPLC purity) of the mixture according to an embodiment of the present disclosure can be calculated from the sum of the peak areas derived from the mixture according to an embodiment of the present disclosure with respect to the peak area by HPLC and UV light of 254 nm (hereinafter, the total HPLC area). Further, similarly, the content ratio of impurities that are by-products (hereinafter, content ratio [%] of impurities) can be calculated from the sum of the peak areas derived from impurities that are by-products with respect to the total HPLC area. In the case of using the mixture according to an embodiment of the present disclosure as a charge transport material of an organic electronic device, the content ratio of impurities is favorably 0 to 5% or less, more favorably 0 to 2% or less, and still more favorably approximately 0 to 1.2% or less with respect to the total HPLC area.

An increase in the content ratio [%] of impurities tends to reduce the drive voltage and reduce the effect of improving light emission efficiency without improving a charge injection property, for example, when the mixture according to an embodiment of the present disclosure is used as a charge transport layer of an organic EL device. That is, when the content ratio [%] of impurities is within the range of the content ratio described above, it is conceivable that an organic electronic device in which the mixture according to an embodiment of the present disclosure is used as a charge transport material provides a device that has excellent current efficiency, a low initial voltage, and electrical properties such as an excellent drive life property.

The present disclosure is characterized by a mixture, including the compounds represented by the general formula (1), the general formula (2), and the general formula (3).

In the mixture according to an embodiment of the present disclosure, the content ratio calculated by the HPLC area ratio of the compound represented by the general formula (1) is favorably 18 to 30%, more favorably 20 to 29%, and still more favorably 21 to 27%.

In the mixture according to an embodiment of the present disclosure, the content ratio calculated by the HPLC area ratio of the compound represented by the general formula (2) is favorably 20 to 32%, more favorably 22 to 30%, and still more favorably 23 to 29%.

In the mixture according to an embodiment of the present disclosure, the content ratio calculated by the HPLC area ratio of the compound represented by the general formula (3) is favorably 45 to 55%, more favorably 46 to 54%, and still more favorably 47 to 53%.

### Charge Transport Material

The mixture according to an embodiment of the present disclosure can be used as a charge transport material. In the case of using the mixture according to an embodiment of the present disclosure as a charge transport material, the mixture according to an embodiment of the present disclosure may be contained alone, a known organic semiconductor material may be arbitrarily combined and contained, and other components such as an additive may be contained.

The “charge transport material” used herein means a compound, a mixture, or other compositions that receive charges from an electrode and facilitates the transfer of the charges. The hole transport material is capable of receiving holes from an anode and transporting the holes. The electron transport material is capable of receiving electrons from a cathode and transporting the electrons.

The charge transport material according to an embodiment of the present disclosure is suitable for a charge transport layer of an organic electronic device as one of the applications, and it is possible to deposit and use the charge transport material. At the time of deposition, the charge transport material needs to have solubility in the above-mentioned organic solvent and excellent processability.

### Solution Process

The mixture according to an embodiment of the present disclosure has solubility suitable for a solution process. In particular, the mixture according to an embodiment of the present disclosure can be used in a solution state as a composition in a solution process to produce an organic electronic device. The “solution process” used herein is a process of easily preparing a multilayer structure device by applying a solution obtained by dissolving a charge transport material and the like in an organic solvent or the like, using a from (composition) such as a dispersion and an emulsion.

In the case of using a charge transport material in a solution process, also the method of depositing the charge transport material is not particularly limited, and the charge transport material can be deposited using a known method. Examples of the method include coating methods such as casting, spin coating, dip coating, blade coating, wire bar coating, and spray coating, printing methods such as inkjet printing, screen printing, offset printing, and relief printing, and soft lithography methods such as a microcontact printing method.

In the present disclosure, examples of the solvent used at the time of deposition include, but not United to, aromatic organic solvents such as benzene, toluene, xylene, mesitylene, tetralin (1,2,3,4-tetrahydronaphthalene), monochlorobenzene, o-dichlorobenzene, m-dichlorobenzene, p-dichlorobenzene, and nitrobenzene; alkyl halide organic solvents such as dichloromethane, chloroform, 1,2-dichloroethane, 1,1,2-trichloroethane, and dichloromethane; nitrile solvents such as benzonitrile and acetonitrile; ether solvents such as tetrahydrofuran, dioxane, diisopropylether, c-pentylmethylether, ethylene glycol dimethylether, ethylene glycol diethylether, and propylene glycol monomethylether; ester solvents such as ethyl acetate and propylene glycol monomethyl ether acetate; and alcohol solvents such as methanol, isopropanol, n-butanol, propylene glycol, 1,3-butanediol, 1,4-butanediol, 2,3-butanediol, cyclohexanol, and 2-n-butoxyethanol. Further, the solvents described above may be used alone or two or more of them may be mixed and used, and the solvent to be used can be selected depending on the structure.

### Evaluation of Solubility

The mixture according to an embodiment of the present disclosure tends to have improved solubility in the organic solvent described above, Regarding the solubility of the mixture according to an embodiment of the present disclosure, the mixture according to an embodiment of the present disclosure is added to 10 g of an organic solvent such as tetrahydrofuran (THF) and toluene, the obtained mixture is stirred for 3 to 5 minutes at room temperature (25±5° C.), and then, the solubility (or saturated solubility) is visually evaluated.

In order to provide the solubility necessary for an industrial operation of a solution process, the solubility of the mixture according to an embodiment of the present disclosure described above is favorably 20% (w/w %) or more, more favorably 40% (w/w %), and still more favorably 50% (w/w %) or more.

### Evaluation of Mobility

The mobility is a quantity indicating the ease of charge transfer of electrons or holes in a solid substance when the mixture according to an embodiment of the present disclosure is used as a charge transport material in an organic electronic device. A mobility 82 (cm²/V·s) is an average travelling speed v of charges divided by an electric field strength E, and can be considered as a proportional coefficient when the charges are accelerated in the presence of an electric field. Since the mobility is inversely proportional to the resistivity in semiconductors, the mobility is an important parameter that determines the electrical properties of a substance.

In the Time-of-Flight method (TOF method) that is a general method for obtaining the drift mobility of charges in the bulk, the average speed v of charges is measured focusing on the mobility being a proportional coefficient when the charges are accelerated in the presence of an electric field. The average travelling speed v of charges can be obtained by measuring a time t_{T} (transit time) necessary for the charges to pass through a sample with a sample thickness d and a voltage V, and the carrier mobility μ can be calculated by dividing the obtained average speed v of charges by the electric field strength E. First, a constant electric field (E=V/L[V/cm]) is applied to a distance between electrodes (d[cm]) and optical carriers are injected by applying pulsed light to the vicinity of one of the electrodes. As a result, only carriers having the same polarity as that of the electric field applied to the electrode travel toward the counter electrode. In this regard, the travelling time ([sec]) between the electrodes is measured. The change with time of the current was represented by a log-log graph and the transit time (t_{T}, [sec]) was obtained on the basis of the change in the slope. The drift mobility (μ) is calculated using the following formula (a-1).

$$μ = \frac{v}{E} = \frac{d}{E \cdot {t}_{T}} = \frac{{d}^{2}}{V \cdot {t}_{T}} ( a - 1 ) \qquad [ Math . 1 ]$$

### Organic Electronic Device

Next, an organic electronic device according to an embodiment of the present disclosure will be described.

The organic electronic device according to an embodiment of the present disclosure is characterized by being formed using the above-mentioned charge transport material according to an embodiment of the present disclosure. The type of the organic electronic device is not particularly limited as long as the charge transport material according to an embodiment of the present disclosure can be applied. Examples of the type of the organic electronic device include an organic EL device, an organic thin film transistor (field effect transistor (FET)), and a photoelectric conversion device used in an optical sensor and a solar battery.

It is known that the organic electronic device includes another layer. Further, the organic electronic device can be prepared by sequentially depositing individual layers on a suitable substrate or using a solution process or the like. Examples of the substrate include an inorganic substrate formed of glass or the like and a plastic substrate formed of a polymer. Specific examples thereof include polyester, polycarbonate, polyimide, polyether sulfone, amorphous polyolefin, epoxy resin, polyamide, polybenzoxazole, and polybenzothiazole. Further, the organic electronic device can be prepared by a combination of vapor deposition and application of individual layers.

The charge transport material according to an embodiment of the present disclosure can be used alone, of course, but can also be mixed with another material and used. Further, the charge transport material according to an embodiment of the present disclosure can be used as a laminated structure with another layer.

### Organic EL Device

An embodiment of an organic electronic device using the mixture according to an embodiment of the present disclosure includes, for example, an organic EL device. In contrast to known compounds such as N,N′-di(naphthalene-1-yl)-N,N′-diphenyl-benzidine (hereinafter, NPD) and 4,4′-bis[N-(3-methylphenyl)-N-phenylamino]biphenyl (hereinafter, TPD) that use a deposition process and are usually used as a hole transport material in the production of an organic EL device, the mixture according to an embodiment of the present disclosure exhibits high solubility in a solvent normally used in a solution process.

The basic structure of an organic EL device according to an embodiment of the present disclosure includes an anode (transparent electrode), a photoactive layer, and a cathode (counter electrode).

An organic EL device that includes, in a photoactive layer, at least one layer containing the mixture according to an embodiment of the present disclosure or a composition containing the mixture can be provided. The mixture according to an embodiment of the present disclosure can be provided as a single layer or a plurality of layers in a photoactive layer disposed between an anode (transparent electrode) and an electrode of a cathode and can be used as particularly a charge transport layer, a hole transport layer, or a light-emitting layer but is not limited thereto. Other layers can be produced with arbitrary known materials.

The anode (transparent electrode) is an electrode that is particularly efficient for injecting holes. For example, the anode can be formed of a metal, a mixed metal, an alloy, a metal oxide, a mixed metal oxide, a conductive polymer, a combination thereof, or a conductive material containing the mixture thereof. In the case where a conductive material needs to have translucency, examples of the conductive material include a conductive transparent oxide semiconductor such as tin-doped indium oxide (ITO), fluorine-doped tin oxide (FTO), and an indium-tin composite oxide.

As the light-emitting layer in the photoactive layer, a known material can be used in addition to the mixture according to an embodiment of the present disclosure. In addition to a metal complex of a quinolinol derivative including tris(8-hydroxyquinolinato)aluminium (hereinafter, Alq₃), various metal complexes, an anthracene derivative, a bis-styrylbenzene derivative, a pyrene derivative, an oxazole derivative, a polyparaphenylene vinylene derivative, and the like can be used. Further, the light-emitting layer may be formed of a host material and a dopant material. As the host material, in addition to the light-emitting material, a thiazole derivative, a benzinidazole derivative, a polydialkylfluorene derivative, and the like can be used. Further, as the dopant material, quinacridone, coumarin, rubrene, perylene, derivatives thereof, a benzopyran derivative, a rhodamine derivative, an aminostyryl derivative, and the like can be used. These materials can be formed into a thin film by a known method such as a spin coat method and an ink jet method in addition to a vapor deposition method.

Examples of the structure of the organic EL device include one in which an anode, a hole transport layer, ar. electron blocking layer, a light-emitting layer, an electron transport layer, and a cathode are sequentially provided on a substrate, one in which a hole injection layer is provided between an anode and a hole transport layer, one in which an electron injection layer is provided between an electron transport layer and a cathode, and one in which a hole blocking layer is provided between a light-emitting layer and an electron transport layer. In these multilayer structures, several organic layers can be omitted. For example, an anode, a hole transport layer, a light-emitting layer, an electron transport layer, and a cathode may be sequentially provided on substrate

In an organic EL device, charges injected from both electrodes are recombined in the light-emitting layer to obtain light emission. It is important how efficiently both charges of the hole and the electron can be transferred to the light-emitting layer, and the charge transport material plays an important role. For example, in the hole transport layer, by enhancing the hole injection property and electron blocking performance, the probability of recombination of holes and electrons can be improved and high light emission efficiency can be achieved.

As the hole transport layer of the organic EL device, N,N′-diphenyl-N,N′-di(m-tolyl)benzidine (hereinafter, abbreviated as TPD), N,N′-diphenyl-N,N′-di(α-naphthyl)benzidine (hereinafter, abbreviated as NPD), and the like can be used in addition to the mixture according to an embodiment of the present disclosure. Further, as the hole injection/transport layer, a coating type polymer material such as poly(3,4-ethylenedioxythiophene) (hereinafter, abbreviated as PEDOT)/poly(styrene sulfonate) (hereinafter, abbreviated as PSS) can be used.

As the hole blocking layer of the organic EL device, compounds such as various rare earth complexes, a triazole derivative, a triazine derivative, and an oxadiazole derivative can be used in addition to a metal complex of a quinolinol derivative such as a phenanthroline derivative such as bathocuproine and aluminum (III) bis(2-methyl-8-quinolinate)-4-phenylphenolate (hereinafter, abbreviated as BAlq). These materials may also serve as the material of the electron transport layer.

As the electron transport layer of the organic EL device, various metal complexes, a triazole derivative, a triazine derivative, an oxadiazole derivative, a thiadiazole derivative, a carbodiimide derivative, a quinoxaline derivative, and the like can be used in addition to a metal complex of a quinolinol derivative including Alq₃ and BAlq.

As the electron injection layer of the organic EL device, an alkali metal salt such as lithium fluoride and cesium fluoride, an alkaline earth metal salt such as magnesium fluoride, and a metal oxide such as aluminum oxide can be used, but this can be omitted.

Specific examples of the material used as the cathode (counter electrode) of the organic EL device include metals such as platinum, titanium, stainless, aluminum, gold, silver, and nickel, and alloys thereof. An electrode material having a low work function, such as aluminum, or an alloy having a lower work function, such as a magnesium silver alloy, a magnesium indium alloy, and an aluminum magnesium alloy can be used as an electrode material.

Other examples of the organic electronic device that includes at least one layer containing the mixture according to an embodiment of the present disclosure or a composition containing the mixture include, but not limited to, a solar battery and an optical sensor including a photoelectric conversion device.

### Photoelectric Conversion Device

An embodiment of the organic electronic device using the mixture according to an embodiment of the present disclosure is, for example, a photoelectric conversion device. The basic structure of a photoelectric conversion device according to an embodiment of the present disclosure includes a transparent electrode (conductive support), a photoelectric conversion unit, and a counter electrode.

The structure of the photoelectric conversion device favorably includes, but not limited to, a conductive support, a hole blocking layer, an electron transport layer, a photoelectric conversion layer, a hole transport layer, and a counter electrode on a substrate sequentially. Further, a conductive support, a hole transport layer, a photoelectric conversion layer, an electron transport layer, and a counter electrode may be provided in the stated order.

As the conductive support of the photoelectric conversion device and both electrodes of the counter electrode, the materials of the anode and the cathode of the organic EL device described above can be similarly used.

Specific examples of the semiconductor forming an electron transport layer include metal oxides such as tin oxide (SnO, SnO₂, SnO₃, and the like), titanium oxide (TiO₂ and the like), tungsten oxide (WO₂, WO₃, W₂O₃, and the like), zinc oxide (ZnO), niobium oxide (Nb₂O₅ and the like), tantalum oxide (Ta₂O₅ and the like), yttrium oxide (Y₂O₃ and the like), strontium titanate (SrTiO₃ and the like); metal sulfides such as titanium sulfide, zinc sulfide, zirconium sulfide, copper sulfide, tin sulfide, indium sulfide, and tungsten sulfide; metal selenides such as titanium selenide, zirconium selenide, and indium selenide; and element semiconductors such as silicon. The electron transport layer can be obtained using a known deposition method in accordance with the forming material. As the method of depositing an electron transport layer, an arbitrary coating method for coating with a coating liquid can be used.

For example, in the case of using a perovskite material that is a photoelectric conversion layer as a perovskite type photoelectric conversion device, a layer containing a perovskite material of a mixed cation or a mixed anion represented by an arbitrary composition such as methylammonium PbI₃, formamidinium PbI₃, ethylammonium PbI₃, and CsPbI₃ can be used as an example, but the present disclosure is not limited thereto. It is favorable to use one or two or more kinds of these perovskite materials and a light absorber other than the perovskite material may be contained.

The mixture according to an embodiment of the present disclosure is useful as a charge transport material of a photoelectric conversion device, or the like. In particular, in the case of using the mixture according to an embodiment of the present disclosure as a hole transport material, it is possible to efficiently take out a current and obtain a photoelectric conversion device with high efficiency and high durability.

In the case of using the mixture according to an embodiment of the present disclosure as a hole transport layer of a photoelectric conversion device, an additive may be contained for the purpose of further improving the hole transport property. As an additive for the hole transport layer, a dopant (or an oxidizing agent) or a basic compound (or a basic additive) may be contained. Improving the carrier concentration (doping) of the hole transport material in the hole transport layer by causing the hole transport layer to contain an additive leads to improvement in the conversion efficiency of the photoelectric conversion device.

The photoelectric conversion device using the charge transport material according to an embodiment of the present disclosure is applicable to a dye-sensitized solar battery, a perovskite type solar battery, an organic thin film solar battery, various optical sensors, and the like. In the case of using the photoelectric conversion device for a solar battery, a photoelectric conversion device containing the mixture according to an embodiment of the present disclosure is a cell, the necessary number of cells are arranged to obtain a module, and a predetermined electrical wiring is provided, thereby obtaining the solar battery.

### Organic Thin Film Transistor (Field Effect Transistor)

As an embodiment of an organic electronic device according to an embodiment of the present disclosure, a field effect transistor (FET) that is a kind of switching element will be described as an example. The basic structure of the field effect transistor includes an insulator layer, a gate electrode and a charge transport layer isolated by this insulator layer, and a source electrode and a drain electrode provided to be in contact with this charge transport layer on a support substrate. The order in which the respective layers are laminated is not particularly limited, and these layers may be laminated in any order.

The charge transport material according to an embodiment of the present disclosure is appropriately deposited on a substrate or the like and used as a charge transport layer. The film thickness of the charge transport film is not particularly limited. In the case of the field effect transistor illustrated above, the properties of the device do not depend on the film thickness as long as the film thickness is a necessary thickness or more. Therefore, a favorable film thickness is normally 1 nm or more, and favorably 10 nm or more. Further, the film thickness is normally 10 μm or less, and particularly favorably, 500 nm or less.

The organic thin film transistor using the charge transport material according to an embodiment of the present disclosure can be used as a switching element of the active matrix of a display. This performs high-speed and high-contrast display by utilizing the fact that the current between the source and the drain can be switched by the voltage applied to the gate to turn on the switch only when a voltage is applied or a current is supplied to a display device and disconnect the circuit in other times, and is expected as a device capable of performing energy-saving processes and low-cost processes.

Although a favorable embodiment has been described above, the present disclosure is not limited thereto arid may be appropriately modified without departing from the essence of the present disclosure.

### EXAMPLES

Although the present disclosure will be specifically descried below by way of Examples, the present disclosure is not limited to the following Examples. Note that the physical properties and identification of the product obtained in a Synthesis Example were performed by melting point measurement (manufactured by METTLER TOLEDO., model number FP-62) or high performance liquid chromatography (manufactured by SHIMADZU CORPORATION, model number LC-10A). A “part” in the Examples represents a mass part.

### Example 1

(Synthesis of mixture No. 1 containing compound (A-1), compound (A-2), and compound (B-1))

24.25 g (132 mmol) of 4-methyldiphenylamine, 26.10 g (132 mmol) of 4,4′-dimethyldiphenylamine, 46.7 g (115 mmol) of 4,4′-diodobiphenyl, 39.75 g (288 mmol) of anhydrous potassium carbonate, 0.7 g (10 mmol) of a copper powder, 1.2 g (11 mmol) of sodium bisulfite, 0.7 g (2.8 mmol) of 3,5-di-tert-butylsalicylic acid, and 111 mL of toluene were mixed, heated to 220 to 225° C. while introducing a nitrogen gas, and stirred for 6 hours. After the reaction was completed, the reaction product was extracted with 288 ml of toluene, the insoluble matter was removed by filtration, and then, the filtrate was concentrated to dryness. The obtained solid was purified by column chromatography (carrier; silica gel, eluent; toluene:hexane=1:4) to obtain 54.91 g (yield; 88.2%, HPLC purity of 99.3%, melting point; 141 to 143° C.) of a mixture No. 1 of p-diphenyl compound derivatives containing 4,4′-bis(4-methyldiphenylamino)-p-diphenyl (compound (A-1)), 4-(4-methyldiphenylamino)-4′-diphenylamino-p-diphenyl (compound (A-2)), and 4,4′-bis(diphenylamino)-p-diphenyl (compound (B-1)) with a mixing ratio of the compound (A-1):the compound (A-2):the compound (B-1)=23.5:49.7:26.8 (peak area ratio by high performance liquid chromatography)

The identification of a compound was performed by identifying individual compounds with a commercially available product or a single synthetic product and comparing the retention time by high performance liquid chromatography. The measurement conditions of high performance liquid chromatography for identification of a compound and measurement of a mixing ratio are as follows:

Column:OBS column, eluent:tetrahydrofuran/methanol=1/10 (v/v), measurement wavelength:254 nm.

### Measurement of Solubility in Organic Solvent

The mixture No. 1 was put in a transparent sample bottle and added to 10 g of tetrahydrofuran (hereinafter, THF) and 10 g of toluene at room temperature (25±5° C.) and stirred for 3 to 5 minutes, and then the solubility was visually checked. The results are shown in Table 1. The unit of solubility was expressed as % (w/w).

### Example 2

(Synthesis of mixture No. 2 containing compound (A-1), compound (A-2), and compound (B-1))

20.4 g (111 mmol) of 4-methyldiphenylamine, 26.8 g (136 mmol) of 4, 4′-dimethyldiphenylamine, 43.65 g (107.5 mmol) of 4,4′-diodobiphenyl, 37.15 g (269 mmol) of anhydrous potassium carbonate, 0.7 g (10 mmol) of a copper powder, 1.2 g (11 mmol) of sodium bisulfite, 0.7 g (2.8 mmol) of 3,5-di-tert-butylsalicylic acid, and 111 mL of toluene were mixed, heated to 220 to 225° C. while introducing a nitrogen gas, and stirred for 6 hours. After the reaction was completed, the reaction product was extracted with 150 ml of toluene, the insoluble matter was removed by filtration, and then, the filtrate was concentrated to dryness. The obtained solid was purified by column chromatography (carrier; silica gel, eluent; toluene:hexane=1:4) to obtain 52.95 g (yield; 92.8%, HPLC purity of 99.0%, melting point; 167 to 173° C.) of a mixture No.2 of p-diphenyl compound derivatives containing 4,4′-bis(4-methyldiphenylamino)-p-diphenyl (compound (A-1)), 4-(4-methyldiphenylamino)-4′-diphenylamino-p-diphenyl (compound (A-2)), and 4,4′-bis(diphenylamino)-p-diphenyl (compound (B-1)) with a mixing ratio of the compound (A-1):the compound (A-2):the compound (B-1)=18.8:48.4:31.8 (peak area ratio by high performance liquid chromatography).

Identification of the compound was performed in the sane manner as that in the Example 1.

### Measurement of Solubility in Organic Solvent

The solubility measurement was performed in the same manner as that in the Example 1 except that the mixture was changed to the mixture No. 2. The results are shown in Table 1.

### Example 3

(Synthesis of mixture No. 3 containing compound (A-1), compound (A-2), and compound (B-1))

24.9 g (136 mmol) of 4-methyldiphenylamine, 29.5 g (111 mmol) of 4,4′-dimethyldiphenylamine, 43.65 g (107.5 mmol) of 4,4′-diodobiphenyl, 37.15 g (269 mmol) of anhydrous potassium carbonate, 0.7 g (10 mmol) of a copper powder, 1.2 g (11 mmol) of sodium bisulfite, 0.7 g (2.8 mmol) of 3,5-di-tert-butylsalicylic acid, and 111 mL of toluene were mixed, heated to 220 to 225° C. while introducing a nitrogen gas, and stirred for 6 hours. After the reaction was completed, the reaction product was extracted with 150 ml of toluene, the insoluble matter was removed by filtration, and then, the filtrate was concentrated to dryness. The obtained solid was purified by column chromatography (carrier; silica gel, eluent; toluene:hexane=1:4) to obtain 52.25 g (yield; 92.9%, HPLC purity of 99.2%, melting point; 140 to 142° C.) of a mixture No. 3 of p-diphenyl compound derivatives containing 4,4′-bis(4-methyldiphenylamino)-p-diphenyl (compound (A-1)), 4-(4-methyldiphenylamino)-4′-diphenylamino-p-diphenyl (compound (A-2)), and 4,4′-bis(diphenylamino)-p-diphenyl (compound (B-1)) with a mixing ratio of the compound (A-1):the compound (A-2); the compound (B-1)=28.4:49.2:21.6 (peak area ratio by high performance liquid chromatography).

Identification of the compound was performed in the sane manner as that in the Example 1.

### Measurement of Solubility in Organic Solvent

The solubility measurement was performed in the same manner as that in the Example 1 except that the mixture was changed to the mixture No. 3. The results are shown in Table 1.

### Comparative Example 1

Solubility in organic solvent

The solubility was measured in the same manner as that in Example 1 except that the mixture No. 1 according to the Example 1 was changed to a compound (manufactured by HODGGAYA CHEMICAL CO., LTD.:HCT-306) represented by the following formula (C-1) according to a Comparative Example. The results are shown in Table 1.

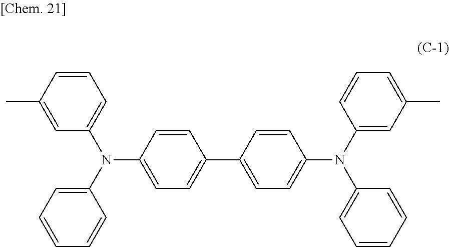

### Comparative Example 2 and Comparative Example 3

Solubility in organic solvent The solubility was measured in the same manner as that in Example 1 except that the mixture No. 1 according to the Example 1 was changed to a compound alone (manufactured by HODOGAYA CHEMICAL CO., LTD.:HCT-308) represented by the following formula (A-1) according to a Comparative Example or a compound alone (manufactured by MITSUBISHI PAPER MILLS LIMITED.:MPCT-61) represented by the following formula (A-2) that is a comparative compound. The results are shown in Table 1.

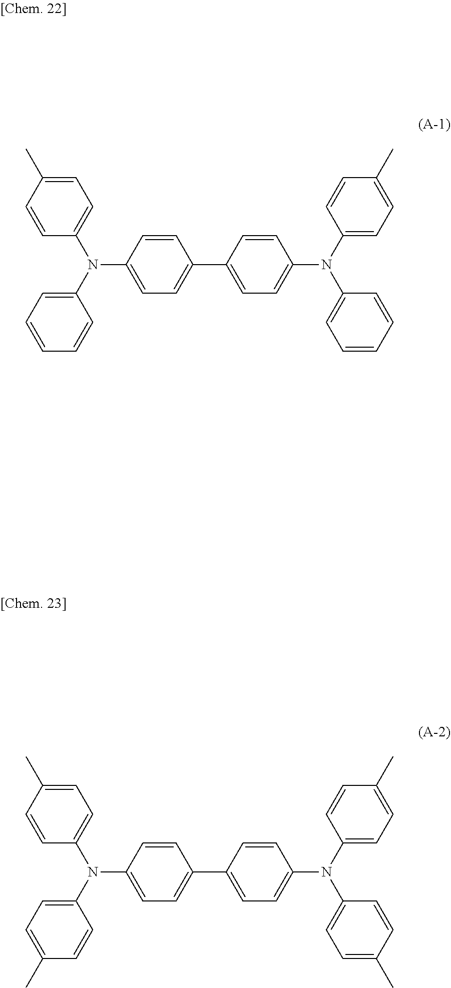 [SMILES: Cc1ccc(N(c2ccccc2)c2ccc(-c3ccc(N(c4ccccc4)c4ccc(C)cc4)cc3)cc2)cc1 ; Cc1ccc(N(c2ccc(C)cc2)c2ccc(-c3ccc(N(c4ccc(C)cc4)c4ccc(C)cc4)cc3)cc2)cc1]

TABLE 1

| | | Solubility | Solubility in |
|---|---|---|---|
| | | in THF [%] | toluene [%] |
| Example 1 | Mixture No. 1 | 147 | 94.2 |
| Example 2 | Mixture No. 2 | 139. 2 | 90.3 |
| Example 3 | Mixture No. 3 | 168.4 | 109.2 |
| Comparative | Comparative compound | 25.3 | 16.1 |
| Example 1 | (C-1) | | |
| Comparative | Comparative campound | 49.9 | 24.2 |
| Example 2 | (A-1) alone | | |
| Comparative | Comparative compound | 38.7 | 17.7 |
| Example 3 | (A-2) alone | | |

When the solubility of each of the mixtures according to an embodiment of the present disclosure and the solubility of each of the comparative compounds were compared with each other, it was confirmed that the mixture according to an embodiment of the present disclosure showed the solubility 3 to 7 times those of the comparative compounds and had excellent solubility.

### Example 4

The mixture No. 1 (1.0 part) obtained in the Example 1 was added to 12.2% tetrahydrofuran of a polycarbonate resin (Iupilon Z, manufactured by Mitsubishi Engineering-Plastics Corporation) and dissolved by applying ultrasonic waves. The obtained solution was applied onto an aluminum surface of an aluminum-deposited PET film that is a conductive support with a wire bar and dried at 110° C. under normal pressure for 30 minutes to form a film having a film thickness of 10 μm. Further, a translucent gold electrode was deposited on the film. A dye laser having a pulse width of 3 nsec and a center wavelength of 610 nm was applied to a thin film via the translucent gold electrode while applying a voltage between the conductive support of a measurement sample and the translucent gold electrode. The drift mobility of the obtained device was measured by a Time-of-flight method with an electric field strength of 2×10⁵ (V/cm). The results are shown in Table 2.

### Comparative Example 4

A device was prepared in the same manner as that in the Example 4 except that a compound (manufactured by KODOGAYA CHEMICAL CO., LTD.:HCT-305N) represented by the following formula (A-7) was used as a comparative compound, and the drift mobility of the prepared device was measured. The results are shown in Table 2.

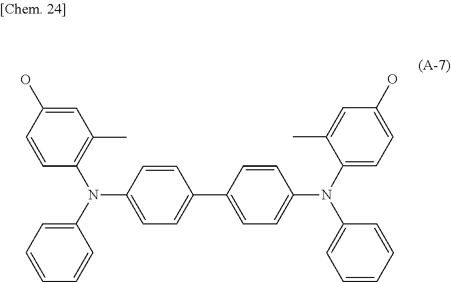

TABLE 2

| | Drift mobility |
|---|---|
| | [cm²/V · s] |
| Example 4 | 2.2 × 10⁻⁵ |
| Comparative | 8.0 × 10⁻⁸ |
| Example 4 | |

From Tables 1 and 2, it can be seen that the mixture of p-diphenyl compound derivatives according to an embodiment of the present disclosure has higher solubility in an organic solvent than the existing products and has excellent drift mobility.

Since the mixture of p-diphenyl compound derivatives according to an embodiment of the present disclosure has clearly improved solubility in an organic solvent as compared with the existing products and has excellent mobility to exhibit favorable properties of an organic semiconductor material, the mixture of p-diphenyl compound derivatives is useful as a charge transport material capable of realizing applications to a photoelectric conversion device such as a solar battery and an optical sensor, an organic EL device, and an organic electronic device such as an organic thin film transistor.

It should be understood by those skilled in the art that various modifications, combinations, sub-combinations and alterations may occur depending on design requirements and other factors insofar as they are within the scope of the appended claims or the equivalents thereof.

1. A mixture containing p-diphenyl compound derivatives represented by the following general formula (1), general formula (2), and general formula (3). 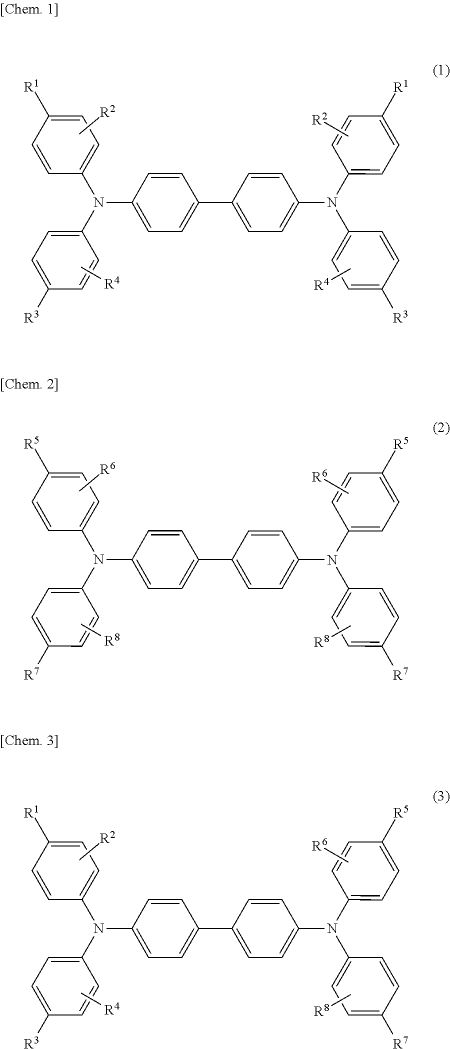
[In the formula, R¹ to R⁸ independently represent a hydrogen atom, a halogen atom,
a linear or branched alkyl group having 1 to 20 carbon atoms, which may have a substituent group,
a linear or branched alkenyl group having 2 to 20 carbon atoms, which may have a substituent group,
a cycloalkyl group having 3 to 10 carbon atoms, which may have a substituent group,
a linear or branched alkoxy group having 1 to 20 carbon atoms, which may have a substituent group,
a cycloalkoxy group having 3 to 10 carbon atoms, which may have a substituent group,
an amino group having 1 to 20 carbon atoms, which may have a substituent group, or
an aromatic hydrocarbon group having 6 to 36 carbon atoms, which may have a substituent group, and
R¹ and R², R³ and R⁴, R⁵ and R⁶, and R⁷ and R⁵ may each be bonded to each other to form a ring.
However, the type and/or substation position of at least one of the substituent groups R⁵ to R⁸ is different from those of the substituent groups R¹ to R⁴. One of R¹ and R³ and one of R⁵ and R⁷ are not hydrogen atoms.]

2. The mixture according to claim 1, wherein
in the general formula (1), the general formula (2), and the general formula (3), R¹, R³, R⁵, and R⁷ each represent a hydrogen atom, a linear or branched alkyl group having 1 to 20 carbon atoms, which may have a substituent group, or a linear or branched alkoxy group having 1 to 20 carbon atoms, which may have a substituent group.

3. The mixture according to claim 1, wherein a content ratio of the compound represented by the general formula (1) is 18 to 30%.

3. The mixture according to claim 3, wherein
a content ratio of the compound represented by the general formula (2) is 20 to 32%.

5. The mixture according to claim 4, wherein a content ratio of the compound represented by the general formula (3) is 45 to 55%.

6. The mixture according to claim 1, wherein
solubility in 100 g of an organic solvent at room temperature (25+5° C.) is 50 weight % or more.

7. A method of producing a mixture containing p-diphenyl compound derivatives represented by the general formula (1), the general formula (2), and the general formula (3) which are obtained by a one-step reaction from compounds represented by the following general formula (4), general formula (5), and general formula (6). 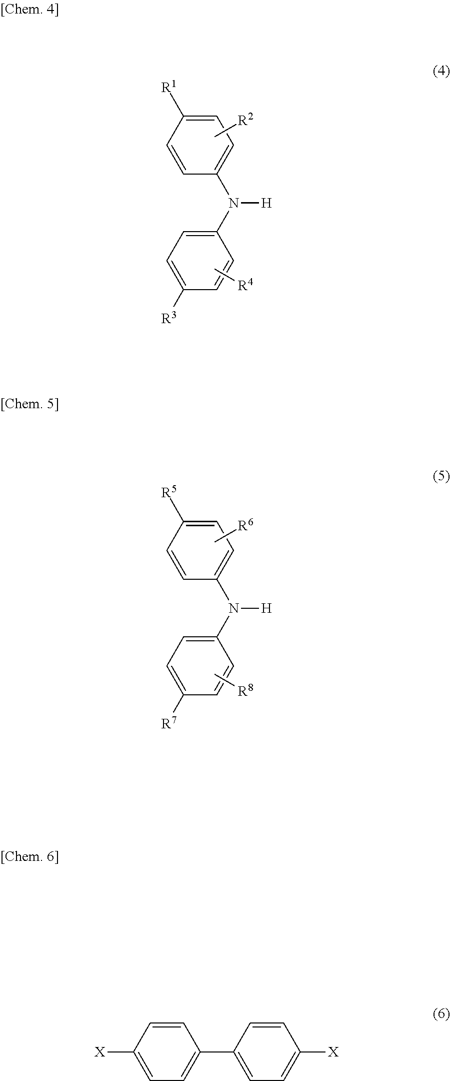
[In the formula, X represents a halogen atom.]

8. The method of producing a mixture according to claim 7, wherein
in the general formula (4) and the general formula (5), R₁, R₃, R₅, and R₇ each represent a hydrogen atom, a linear or branched alkyl group having 1 to 20 carbon atoms, which may have a substituent group, or a linear or branched alkoxy group having 1 to 20 carbon atoms, which may have a substituent group.

9. The method of producing a mixture according to claim 7, wherein
the one-step reaction is an Ullmann reaction and a reaction temperature is 190 to 235° C.

10. The method of producing a mixture according to claim 9, wherein
an additive for the Ullmann reaction is an aromatic oxycarboxylic acid compound.
